(19) United States
(12) Patent Application Publication
(10) Pub. No.: US 2019/0160079 A1
(43) Pub. Date: May 30, 2019

(54) USE OF A BISPHOSPHONATE SALT IN A MEDICAMENT FOR A NATURALLY OCCURRING DISEASE/DISORDER IN A MAMMAL

(71) Applicants: Berendina Oudshoorn, Leiden (NL); Franciscus Cornelis Hilke van Toor, Leiden (NL)

(72) Inventors: Berendina Oudshoorn, Leiden (NL); Franciscus Cornelis Hilke van Toor, Leiden (NL)

(21) Appl. No.: 16/305,021

(22) PCT Filed: May 29, 2017
(86) PCT No.: PCT/NL2017/050344

§ 371 Date: Nov. 27, 2018

(30) Foreign Application Priority Data
May 27, 2016 (NL) 2016848

(51) Int. Cl. A61K 31/663; A61K 9/00; A61P 19/02

(52) U.S. Cl. CPC A61K 31/663; A61P 19/02; A61K 9/0019

(57) ABSTRACT

The present invention is in the field of a use of a bisphophosphonate acid, specifically a salt thereof, or analogue thereof, or combinations thereof, in a medicament for naturally occurring diseases/disorders in a mammal, the mammal being selected from the orders of Perissodactyla, such as the family of Equidae (horses), (Cet) Artiodactyla, such as the family of Bovidae (cattle), and Carnivora, such as the families of Canidae (e.g. dogs) and Felidae (e.g. cats).

### FIELD OF THE INVENTION

The present invention is in the field of a use of a bisphophosphonate acid, specifically a salt thereof, or analogue thereof, or combinations thereof, in the manufacture of a medicament for naturally occurring diseases/disorders in a mammal, the mammal being selected from the orders of Perissodactyla, such as the family of Equidae (horses), (Cet) Artiodactyla, such as the family of Bovidae (cattle), and Carnivora, such as the families of Canidae and Felidae.

### BACKGROUND OF THE INVENTION

The present invention is in the field of a use of a bisphophosphonate acid, or salt thereof, or analogue thereof, or combinations thereof, in a medicament for a naturally occurring diseases/disorders in a mammal.

Osteoarthritis (OA) is considered the most common form of arthritis worldwide and causes not only damage to bone and cartilage in joints of affected human beings, but also mammals such as dogs, cats and horses can be affected in a similar way.

Although the terms “degenerative joint disease” and “osteoarthritis” are commonly used interchangeably in the veterinary arena, a distinction has been made between the two [1]. Degenerative joint disease (DJD) is considered a general term used to describe any degenerative change in a synovial, cartilaginous, or fibrous articulation in the skeleton. Osteoarthritis, however, is a pathologic change of a diarthrodial synovial articulation and includes deterioration of articular cartilage, osteophyte formation, bone remodeling, soft tissue changes, and low-grade nonpurulent inflammation.

When OA is diagnosed in pets, often owners feel true empathy. They want to treat their pets with all the possibilities, such as those being available for humans. It is noted that e.g. in Western Europe there are around 43 million pet dogs and the total number of dogs in the Netherlands is in excess of 2.2 million. OA is the most prevalent joint disorder in dogs and not just linked to older dogs. It has been found that 20 percent of adult and 85 percent of the geriatric (>8 years old) dogs are affected with OA and may suffer pain and disability [2]. The onset of primary OA is found to depend on various factors, such as breed; all breeds can be affected, but large and giant breeds show a higher prevalence [3, 4]. For instance, in Labrador Retrievers aged >8 years, OA in several joints (elbow, shoulder, hip, knee) is typical. The high prevalence of OA in the canine population makes the disease a major cause of concern.

In many countries the cat is the most popular pet. It is noted that there are e.g. approximately 45 million cats in Europe and 4 million in the Netherlands alone. The feline population is also an ageing one and many of the diseases associated with advancing years, like OA, are associated with chronic pain [5]. OA in cats appears to be much more common than previously thought and could be a major cause of discomfort, especially in senior (>10 years of age) cats. Joint pain in the cat is seldom associated with overt lameness [6, 7]. Changes in the cat's usual behaviour, lifestyle, or both should alert the vet that the animal might be suffering arthritic pain [8, 9]. The Thoracic spine shoulders, elbow and hip are the most frequently affected joints [10] and approximately 50% of cases resemble OA with progressive degeneration of multiple joints.

The distortion between clinical and radiographic finding in dog OA is well-recognized [2], however several clinical studies have reported that the progression of radiographic score is in contrast to the clinical signs [11]. It is noted that radiographic changes are poorly correlated to clinical lameness in cats [6].

Most treatments are focused around pain relief (by dietary supplements and NSAIDs), as no robust treatments have been discovered that slow down the progression of the disease [12, 13].

For human beings a medicament comprising bisphosphonates is known. The development of bisphosphonates is based on studies in the 1960s on the mechanism of mineralization. A non-medical use was related to soften water in irrigation systems used in orange groves [14, 15, 16]. Bisphosphonates can be broadly classified into two major classes with distinct mechanisms of action: the non-nitrogen containing class and the nitrogen-containing class. The earlier non-nitrogen containing bisphosphonates (e.g., Clodronate, Tiludronate and Etidronate) were developed in the 1970s and were found to act by incorporation into ATP. These structurally more simple molecules without nitrogen become toxic to the osteoclasts and hence are therefore discouraged for use. Despite this effect studies have been conducted using tiludronate on dogs (Moreau et al., in “Tiludronate treatment improves structural changes and symptoms of osteoarthritis in the canine anterior cruciate ligament model”, Biomed Central, Arthritis Research & Therapy, 2011, Vol. 13, No. 3, p. R98) and on horses (Delguste et al. in “Pharmacologie clinique des bisphosphonates: revue de littérature axée sur le tiludronate chez le cheval.”, Ann. Méd. Vét., 2007, 151, 269-280); Delguste discourages the use other bisphosphonates, such as of nitrogen containing bisphosphonates. The newer, much more potent nitrogen-containing, bisphosphonates (including Alendronate) are considered to act by inhibiting Farnesyl pyrophosphate synthase (FDPS) in the mevalonate pathway [17]. The presence of a nitrogen molecule in the side chain of the bisphosphonate structure increases its potency. The use of bisphosphonates in the treatment of bone metabolic disorders is known in principle. In the 1990s the actual mechanism of action of bisphosphonates was demonstrated with the initial launch of Fosamax (Alendronate) for human treatment [18, 19].

Alendronate (ALN) is recommended for skeletal disorders for human beings characterized by an increased and abnormal bone remodeling. Subchondral bone is found to play an important role in the pathophysiology of OA and healthy subchondral bone protects cartilage. ALN prevented increased bone turnover and preserved the structural integrity of subchondral bone in experimental OA [20].

ALN therapy might dampen disease progression and less severity of pain is observed.

Despite all the animal models, primary OA as well as secondary OA are not the same as experimentally induced osteoarthritis. Most animal models of osteoarthritis involve chemical or surgical initiation of the disease, whereas the majority of OA is considered primary or secondary. There is however no one gold standard model to represent primary etiology. Each mode of induction and species has distinct advantages and disadvantages, and there are still many gaps in the knowledge of the disease and the relationship between documented degenerative molecular changes and functional and clinical deterioration [21]. A valid concern with these induced models is that while they may be reflective of inflammatory or posttraumatic OA, they are typically not representative of spontaneous, naturally occurring OA [22]. Further examples of animal models can be found in US 2007/004681 A1 (and likewise WO 2005/107751 A) wherein such a model is used for Sprague-Dawley rats; the document is in addition very generic and silent on effects on other animals, on specific diseases/disorders, advantages of certain compounds over others, etc.

In addition, the symptom modifying and structure-modifying effects of bisphosphonates for osteoarthritis remain controversial. Some researchers claim that bisphosphonates are effective in the treatment of OA, but not all bisphosphonates are effective at slowing disease progression in these (animal) models. There is little clinical information available from published studies of the effects of bisphosphonates on OA. Meyer J M. et al. showed that Risedronate but not Alendronate slows disease progression in the guinea pig model of primary osteoarthritis [23]. While Davis A J et al. concluded that there is limited evidence that bisphosphonates are effective in the treatment of OA pain [24]. A study by Soto et al. “Bisphosphonates: Pharmacology and Clinical Approach to Their Use in Equine Osteoarticular Diseases”, J. Equine Veterinary Science, June 2014, Vol. 34, Issue 6, Pages 727-737, recite a study on osteoarticular disease therapy on horses, comparing various bisphosphonates. Nitrogen comprising bisphosphonates are discouraged for several reasons for said therapy, such as a release of tumour necrosis factor alpha. In the study various models are used, but results on naturally occurring diseases are thin; in addition relatively high dosages were used.

Human patients with hip osteoarthritis were treated with Alendronate, in a prospective randomized trial [3, 25], for 2 years. The oral dosage Alendronate was prescribed at 35 mg once a week. Twice as many patients were assigned to the Alendronate group compared with the control group to provide for the evaluation of factors influencing the progression of osteoarthritis among the Alendronate group patients. In conclusion, Alendronate treatment had clinical efficacy of pain reduction but failed to show preventive effects for structural progression of hip osteoarthritis. In a larger-scale randomized controlled trial of Risedronate treatment over a 2-year period in patients with knee osteoarthritis, however, no significant differences in symptom improvement or progression of radiographic osteoarthritis were observed between the placebo group and treatment groups [26]. Likewise hip dysplasia in dogs is a topic of WO 2005/107751 A, wherein an effective amount of a bisphosphonate is preferably applied intranasal or transdermal to the dog typically in a single or multiple dosage per day.

Another application relates to bisphosphonates that are administered intravenously only for the treatment of cancer in humans and Alendronate sodium tablets are, for instance, indicated for the treatment of osteoporosis in postmenopausal women [27, 28].

From the above one may conclude that experimentally induced osteoarthritis is not representative of spontaneous, naturally occurring osteoarthritis. Moreover, the symptom- and structure-modifying effects of bisphosphonates for treatment of osteoarthritis are still contentious.

It is noted that certain limitations in the use of bisphosphonates are observed. It is noted that it is oral dosing that is typically applied, either as an aqueous solution or as a tablet. The fraction of the drug that reaches the circulatory system intact after oral dosing is found to be low (less than 1%) and depending on boundary conditions is even lower. In addition part of the dosing rapidly partitions. It is found that the strong negative charge on the two phosphonate moieties limits oral bioavailability. It is further known that an oral dosage of a bisphosphonate, and in particular alendronate, can cause irritation and inflammation of the esophagus. Therefore, it is considered very important that patients do not lie down or recline for at least 30 minutes after taking the medication.

The present invention therefore relates to a use of bisphosphonates, which solves one or more of the above problems and drawbacks of the prior art, providing reliable results, without jeopardizing functionality and advantages.

### SUMMARY OF THE INVENTION

In a first aspect the present invention relates to a use according to claim 1.

It has been found that salts of bisphosphonic acids according to FIG. 1a-i, analogues thereof, and combinations are very suited in a use in the manufacture of a medicament for a naturally occurring disease/disorder in a mammal, slow down the progression of the disease, reduce symptoms, the mammal being selected from the orders of Perissodactyla, such as the family of Equidae (e.g. horses and donkeys), (Cet) Artiodactyla, such as the family of Bovidae (e.g. cattle), Carnivora, such as the families of Canidae (e.g. dogs) and Felidae (e.g. cats). It is noted that the present selection of mammals in fact may be regarded as animals typically kept by humans, as a pet, for pleasure, and for production of protein comprising food and drinks. It is noted that non-amine (primary, secondary or tertiary) comprising bisphosphonates, and non-heterocycle comprising nitrogen comprising bisphosphonates (see FIG. 1a) do not fall under the scope of the present invention; an example thereof is (as identified above) tiludronate. In addition it is noted that the present invention relates to a medicament comprising only at least one bisphosphonate that can be administered as such; on the contrary for instance WO 2005/123130 A2 relies on a combination treatment including a 5-LOX inhibitor.

It is found that treatment with the above compounds and specifically Sodium Alendronate (a nitrogen containing bisphosphonate) can help reduce pain, slow down the disease and maintain joint movement in the mammals mentioned. For this reason inventors considered introduction of e.g. Alendronate as a new veterinary drug for osteoarthritis in mammals, such as horses, donkeys, cows, dogs and cats.

The surprise was even larger in that only small amounts of medicament (e.g. 0.01-1 mg., preferably 0.05-0.5 mg, such as 0.1-0.2 mg. of active compound per kg animal) were sufficient to observe a totally different behavior from the present mammals; before treatment they in general appeared to be unhappy, not inclined to move freely, not energetic, not inclined to play, whereas after treatment an attitude considered “normal” was observed shortly thereafter, typically in 1-4 weeks, i.e. the total opposite of the before. A single (intravenous) treatment is found sufficient (i.e. no further need) for 3-4 months or even at least half a year. A repetition may be given after 4-12 months. Treatment is found beneficial for all age groups and all breeds can have profit. In general it is noted that timing of potential benefit of antiresorptives in OA might be important. The greatest benefit in the prior art is typically seen very early in the disease. So to maintain the normal attitude mode, typically once per 1-12 month, preferably once per 2-6 months, such as once per 3-5 month, or put different 1-6 times per year, preferably 2-6 times per year, such as 3-4 times per year. For both progressed diseases as well as diseases in an initial phase good results were obtained. As a result a happier and fitter pet is obtained.

The medicament is a physiological liquid comprising a dosage of 0.01-1 mg/kg bisphosphonate, wherein the medicament is for sub-cutaneous or intravenous provision.

As indicated above the present medicament is for application once per 1-12 months, such as 2-3 times/year.

Depending a bit on a precise application the medicament may be provided as a salt, as an analogue, or combinations thereof.

Thereby the present invention provides a solution to one or more of the above-mentioned problems.

Advantages of the present description are detailed throughout the description.

### DETAILED DESCRIPTION OF THE INVENTION

In an exemplary embodiment the present salt is of a nitrogen containing bisphosphonic acid (bisphosphonate) according to FIG. 1a

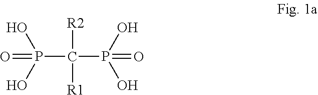

wherein R1 is independently selected from H and OH,
wherein R2 is independently selected from branched, unbranched, aromatic, non-aromatic cyclic, carbon comprising moieties having 1-12 carbon atoms and at least one primary amine, secondary amine, tertiary amine, or heterocycle comprising nitrogen (hence C₁-C₁₂Nₓ),
an analogue of said bisphosphonate, and combinations thereof for use in a medicament for a naturally occurring diseases/disorders in a mammal, the mammal being selected from the orders of Perissodactyla, such as the family of Equidae (horses), (Cet) Artiodactyla, such as the family of Bovidae (cattle), Carnivora, such as the families of Canidae and Felidae.

In an exemplary embodiment of the present salt the carbon comprising moiety is selected from C₂-C₆ branched or un-branched alkyls, such as methyl, ethyl, propyl, butyl, iso-propyl, iso-butyl, pentyl, tert-butyl, and hexyl, and 5- or 6-ring heterocyclic aromatic moieties comprising one or two nitrogens.

In an exemplary embodiment of the present salt the bisphosphonate is selected from Pamidronate (FIG. 1c), Nerodronate (FIG. 1d), Olpadronate (FIG. 1e), amino-hydroxy-(n-hydroxy-oxido-phosphoryl)-alkyl-phosphonic acid according to FIG. 1b, such as Alendronate (FIG. 1i), Ibandronate (FIG. 1f), Risedronate (FIG. 1g), and Zoledronate (FIG. 1h), preferably Alendronate.

In an exemplary embodiment of the present use the alkyl is selected from C₂-C₆ branched or un-branched alkyls, such as methyl, ethyl, propyl, butyl, iso-propyl, iso-butyl, pentyl, tert-butyl, and hexyl. The alkyl is preferably an unbranched alkyl, and even more preferably propyl, butyl or pentyl. It has been found that these alkyls provide somewhat better results.

In an exemplary embodiment of the present use the salt is an alendronate salt ([4-amino-1-hydroxy-1-(hydroxy-oxido-phosphoryl)-butyl]phosphonic acid trihydrate) [CAS number 121268-17-5].

In an exemplary embodiment of the present use the salt is a mono-valent salt, selected from Na—, K—, NH₄—, salts and combinations thereof.

The present use is in a medicament for the treatment of one or more of degenerative arthritis, degenerative joint disease, osteoarthrosis, arthritis, and Osteoarthritis.

The present use is in a suitable form for sub-cutaneous or intravenous or intraperiotenal administration. Unexpectedly especially these administrations provide the best results.

In an exemplary embodiment of the present use the medicament can be applied in a dosage of 0.01-1 mg, preferably 0.05-0.5 mg, such as 0.1-0.4 mg per kg (e.g. 0.2 mg/kg) for dogs and cats, and occasionally slightly lower doses (1-5% lower per kg) for horses and cows, etc.

In the present use the medicament can be applied in a single dose. A next dose may be applied after 6-12 months, e.g. a few (1-4) times per year.

In an exemplary embodiment of the present use the mammal is selected form Canidae, Felidae, Bovidae and Equidae.

In an exemplary embodiment of the present use is in a medicament for the treatment of the one or more of osteoarthritis in a joint, such as in an elbow, hip, knee, wrist, carpus, paw, (lower) back, intervertebral joints and discs, spine, neck, sacroiliac joint, especially in such a joint of a canine or feline, sacro-iliacal-joint of a horse, primary arthrosis, such as due to increased age, secondary arthrosis such as due to a disease or disorder or injury, such as due to trauma, damage, wear, obesity, accident, blow or stroke, such as traumatic arthritis, cranial cruciate ligament rupture or injury and osteochondritis dissecans, especially primary arthrosis of a canine or feline, development disorders, and disorders caused by hereditary and environmental influences, such as dysplasia of a joint, such as an elbow, shoulder, wrist, heel and hip, octeochondrosis dissecans (OCD), Los processus coronoïdeus, Los proc.anconeus, and incongruention, especially of a canine or feline, spondylosis deformans, especially of a canine or feline, deviating aggravation due to impairment, especially of a canine or feline, and

inflammation, such as due to Los Processus Coronoideus, especially of a canine or feline.

The invention is further detailed by the accompanying figures, which are exemplary and explanatory of nature and are not limiting the scope of the invention. To the person skilled in the art it may be clear that many variants, being obvious or not, may be conceivable falling within the scope of protection, defined by the present claims.

The invention although described in detailed explanatory context may be best understood in conjunction with the accompanying exemplary embodiments and figures.

### SUMMARY OF THE FIGURES

FIGS. 1a-i show generic and specific structures of the present bisphosphonate.

### DETAILED DESCRIPTION OF THE FIGURES

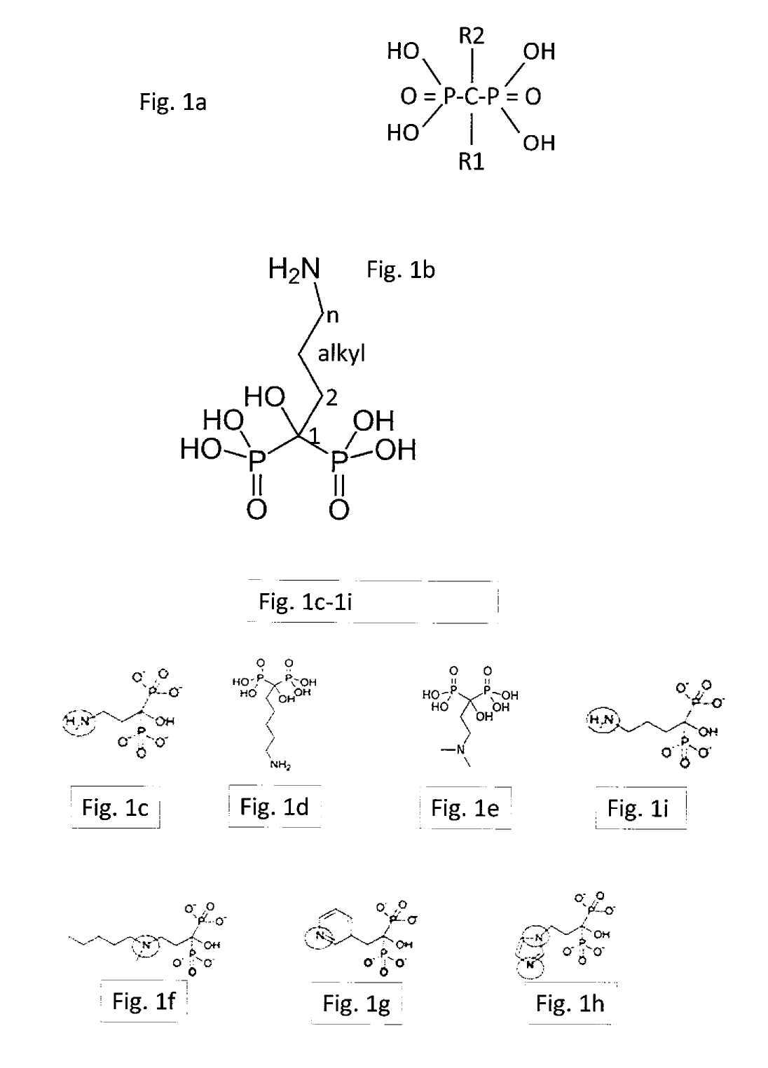

In FIG. 1a a generic structure of a type of bisphosphonates is shown, which bisphosphonates are suited for the present use.

In FIG. 1b an amino-hydroxy-(n-hydroxy-oxido-phosphoryl)-alkyl-phosphonic acid is shown; typically a salt (bisphosphonate) of said acid is used. It comprises two phosphate groups, an alkyl backbone of n carbons, an amino group at a terminus of the alkyl, and a hydroxy group attached to a carbon of the alkyl, whereas the hydroxy-oxido-phosphoryl and phosphonic-acid group are attached to a first carbon of the alkyl.

FIGS. 1c-1i show specific examples of the present bisphosphonates; Pamidronate (FIG. 1c), Nerodronate (FIG. 1d), Olpadronate (FIG. 1e), Ibandronate (FIG. 1f), Risedronate (FIG. 1g), Zoledronate (FIG. 1h) and Alendronate (FIG. 1i). The figures have been detailed throughout the description.

### Experiments

The effect of the present bisphosphonates, and in particular of Pamidronate, Nerodronate, Olpadronate, amino-hydroxy-(n-hydroxy-oxido-phosphoryl)-alkyl-phosphonic acid according to FIG. 1b, Alendronate, Ibandronate, Risedronate, and Zoledronate, and more particularly alendronate, have been tested on various disorders and disease with success. For better understanding of these diseases and disorders, the following section is added, such as to categorize these.

Diseases and Disorders

1 Canines

1a Osteoarthritis

Elbow, hip, stifle (knee), spondylosis (i.e. stress in the joint between the last lumbar vertebra and the sacrum), paws, shoulder, carpus, (wrist), intervertebral discs and intervertebral joints (facet joints), spine, neck, spinal arthritis, sacroiliac luxation, and OA of the sacroiliac joints.

1b Primary Osteoarthritis

An age related disease, seen in older animals in which deterioration of the articular cartilage occurs.

1c Secondary Osteoarthritis

Traumatic arthritis, mechanical abnormalities such as torn menisci, aseptic necrosis, neuroarthropathy, and neoplasia. The inflammatory category is subdivided into an infectious section, which includes articular disease caused by bacterial, fungal, viral, and protozoal organisms, and a noninfectious section, which itself is further divided into two subcategories: immunologic and nonimmunologic.
Cranial cruciate ligament rupture (CCL)
OCD Osteochondritis Dissecans, causes the development of secondary osteoarthritis.

The surface of the joint (the articular cartilage) fails to convert into bone in specific locations. This results in areas of thickened cartilage. These areas are weak and cause the thickened cartilage to detach from the surrounding normal cartilage and form a flap.

It primarily affects the shoulder, elbow, knee (stifle) and ankle (hock) joints. The condition frequently affects both left and right joints (termed ‘bilateral’).

Surgery is generally indicated to remove the fragment of loose cartilage. This can be done arthroscopically or via a direct surgical approach. Following removal of the flap of cartilage the defect heals with an inferior type of cartilage called fibrocartilage.

1d Development Disorders, Hereditary and Environmental Influences

Elbowdysplasia

Elbowdysplasia-research is for 4 different conditions of the elbow-joints, who all eventually will lead to deformation of the joint and will cripple a dog.

The rating of the different parts

The word “Elbowdysplasia” is used when one of the following conditions is present in the elbow-joint:

1. OCD (Osteochondritis dissecans, a loose piece of cartilage from the upper-arm)
2. LPC (Los processus coronoïdeus, a loose piece of bone from the lower forearm)
3. LPA (Los proc.anconeus, a loose piece of bone in any other part of the lower forearm)
4. Incongruention (a not “fitting” joint, due to different lengths from the 2 bones in the lower forearm).

All of the above mentioned conditions will lead to development of osteoarthritis within months. With osteoarthritis, we mean “changes in the joints (bone-reactions) that will develop during the condition.

Hip Dysplasia

1e Spondylosis Defozmans

Spondylosis deformans is a disease of spine in humans and other vertebrates. It occurs when intervertebral discs begin to degenerate, leading to the formation of bony spurs or bridges around the disc and nearby spinal joints

The most common places that spondylosis deformans lesions develop are along the spine, along the thoracic vertebrae (chest), especially at the junction between the rib cage and the abdomen, in the lumbar spine (lower back) and in the lumbosacral spine (around the hips and back legs). In some cases the bony spurs may become large enough that they appear to form a complete bridge between adjacent vertebral bones.

Causes

Repeated microtrauma—repetitive pressure on the same joints, or bones, as through certain exercises or other activities

Major trauma—the body responds by attempting to grow new bone

Inherited predisposition to spurs/congenital bone deformities

Inflammation

2 Felines

2a Osteoarthritis

The most common joints affected either clinically or radiographically are the elbow, hip and knee joints. Shoulder osteoarthritis occurs less frequently in cats than dogs

Elbow, hip, paws, stifle (knee), shoulder, spondylosis (i.e. stress in the joint between the last lumbar vertebra and the sacrum), carpus, (wrist), intervertebral discs and intervertebral joints (facet joints), spine, neck, spinal arthritis, sacroiliac luxation, and OA of the sacroiliac joints.

2b Primary Osteoarthritis

An age related disease, seen in older animals in which deterioration of the articular cartilage occurs.

2c Secondary Osteoarthritis

Traumatic arthritis, mechanical abnormalities such as torn menisci, aseptic necrosis, neuroarthropathy, and neoplasia. The inflammatory category is subdivided into an infectious section, which includes articular disease caused by bacterial, fungal, viral, and protozoal organisms, and a noninfectious section, which itself is further divided into two subcategories: immunologic and nonimmunologic.

Cranial cruciate ligament injury (CCL) occasionally

Abnormal wear on joints and cartilage

Obesity is another factor, as it increases stress on joints.

Dislocation of the knee cap.

OCD Osteochondritis Dissecans, causes the development of secondary osteoarthritis.

Described for the shoulder and stifle (bilateral)

2d Development Disorders, Hereditary and Environmental Influences

A congenital defect present at birth such as an improperly formed hip, also known as hip dysplasia. This can be a cause of joint pain, however, many cats have degenerative changes in their coxofemoral joints on radiographs without signs of discomfort or lameness.

Elbow Dysplasia

2e Spondylosis Deformans

Spondylosis deformans is a disease of spine in humans and other vertebrates. It occurs when intervertebral discs begin to degenerate, leading to the formation of bony spurs or bridges around the disc and nearby spinal joints

In cats, spondylosis deformans lesions are often found along the thoracic spine (along the top of the rib cage/vertebrae of the chest), although they also may develop in the lumbar or sacral areas (lower back). In some cases the bony spurs may become large enough that they appear to form a complete bridge between adjacent vertebral bones

Causes

Repeated microtrauma—repetitive pressure on the same joints, or bones, as through certain exercises or other activities

Major trauma—the body responds by attempting to grow new bone

Inherited predisposition to spurs/congenital bone deformities

Inflammation

3 Horses

Sacroiliac (SI) disease is a recently recognized condition, and veterinarians are seeing more and more performance horses with this issue. The cause of back pain is, however, difficult to pinpoint. The sacroiliac joint is a small articular space between the ventral wing of the ilium and the dorsal wing of the sacrum. On the sacral articular surface, there is hyaline cartilage; on the ilial articular surface, there is fibrocartilage. The sacroiliac joint is typically L shaped, with the convex border directed caudoventrally. The joint holds 0.5 to 1 mL of synovial fluid. The sacroiliac joint attaches the pelvis to the axial skeleton and provides support during weightbearing by transferring propulsive forces of the hindlimb to the vertebral column. The joint is expected to undergo shear forces more than compressive forces, with minimal flexion and extension. Ligaments of the joint include the dorsal sacroiliac ligament, which has dorsal and lateral portions; the interosseous sacroiliac ligament; and the ventral sacroiliac ligament. These ligaments and the sacrosciatic ligament form a sling that attaches the pelvis to the axial skeleton.

The pathology of sacroiliac joint injuries may involve the joint itself or soft tissue structures surrounding it. Osteoarthritis is the most prevalent cause of sacroiliac joint injury, resulting in articular surface lipping, cortical buttressing, articular recession, osteophyte formation, and intraarticular erosion. These problems are often bilaterally symmetric and may be due to chronic instability of the sacroiliac joint. The most common soft tissue injury of the sacroiliac joint is sacroiliac ligament desmitis, which may contribute to joint instability. The dorsal portion of the dorsal sacroiliac ligament seems to be affected most often, showing loss of normal echogenicity and a decrease in parallel fiber pattern on ultrasonography. Disruption of the ligaments increases joint instability, possibly resulting in subluxation and pain.

Subacute or chronic sacroiliac strain and osteoarthrosis of the sacroiliac joint cause typical back soreness. There is often a history of poor performance, with an intermittent, often shifting, hindlimb lameness. This may be associated with some restriction in hindlimb action and dragging of the toes of the hindlimb(s).

Results

The following experiments were performed during November 2015-May 2016. In various veterinarian locations in the Netherlands the animals were treated or occasionally the animals were treated on a farm in a stable.

Cats

To eight cats of ages varying from 12-18 years, typically Felis catus, having osteoarthritis or similar complaints 0.2 mg/kg alendronate was provided in a subcutaneous or intravenous manner. For cats it is preferred to provide the dosage in a subcutaneous manner. Seven cats were free of complaints within a few weeks. Later experiments on more cats showed that more than 75% of the cats showed a good response (being substantially free of complaints). One still had complaints: in this specific case the cat was taking various other medicines, which were refrained from; for this specific cat of course positive effects of the alendronate could not be observed as these were overshadowed by the negative effects of the refrainment.

Dogs

Typical symptoms for dogs and other animals mentioned in the description are lameness, stiffness, especially in the morning, after a stroll and when starting, moving with relatively short steps, a preference for lying down, no stamina, various difficulties/limitations, such as in rising or lying down, in keeping up walking pace, rising a leg, jumping on a couch, tail between the legs and squealing from pain. The present bisphosphonates reduce these symptoms significantly.

To 24 dogs of age varying from 5-15 years, of varying breed, having osteoarthritis or similar complaints 0.20 mg/kg alendronate was provided in an intravenous manner; during about 15 min. a physiological solution comprising alendronate was provided. Complaints varied from light, e.g. noticeable walking/running problems, to serious, e.g. almost not being capable of walking or rising. Especially larger dogs have these types of problems, and typically knees, elbows, hip and back cause problems. All dogs have significantly less complaints or were free of complaints within a few weeks. Such is reflected in feedback from the dog owners.

Horses

To about 100 horses, 4-22 years old, having osteoarthritis in the SI joint 0.2 mg/kg alendronate was provided in an intravenous manner. Most horses were free of complaints for the SI joint within a relative short period of time, some even within a week. For horses in particular, but also for other animals the medicament is preferably provided after the animals are fully grown (adults), which can e.g. be checked by closed epiphysis. For horses especially the SI joint can be treated well.

Donkey

One donkey was treated in line with the horses above. Also results here were fine.

Cows

To 1 bull, of age 6 years, having osteoarthritis 0.2 mg/kg alendronate was provided in an intravenous manner. The bull was free of complaints within a month.

It should be appreciated that for commercial application it may be preferable to use one or more variations of the present system, which would similar be to the ones disclosed in the present application and are within the spirit of the invention.

### LITERATURE CITED

1. Johnston S A. Osteoarthritis. Joint anatomy, physiology and pathobiology. Veterinary Clinics of North America: Small Animal Practice 27 (4): 699-723 (1997)
2. Rialland P et al. Clinical validity of outcome pain measures in naturally occurring canine osteoarthritis. BMC Veterinary Research 8:162-174 (2012)
3. Rychel J K. Diagnosis and treatment of osteoarthritis. Topics in Companion Animal Medicine 25(1): 20-25 (2010)
4. Mele E. Epidemiology of osteoarthritis Veterinary Focus 17 (3): 4-10 (2007)
5. Robertson S A. Osteoarthritis in cats: What we now know about recognition and treatment. Veterinary Medicine November (2008)
6. Clarke S P et al. Prevalence of radiographic signs of degenerative joint disease in a hospital population of cats. Veterinary Record 157: 793-799 (2005)
7. Sul R M et al. Comparison of meloxicam and a glucosamine-chondroitin supplement in management of feline osteoarthritis. Vet Comp Orthop Traumatol 27: 20-26 (2014)
8. Bennett D. et al. A study of owner observed behavioural and lifestyle changes in cats with musculoskeletal disease before and after analgesic therapy. Journal of Feline Medicine and Surgery 11: 997-1004 (2009)
9. Clarke S P et al. Feline osteoarthritis: a prospective study of 28 cases Journal of Small Animal Practice 47: 439-445 (2006)
10. L. I. Slingerland L I et al. Cross-sectional study of the prevalence and clinical features of osteoarthritis in 100 cats. The Veterinary Journal 187: 304-309 (2011)
11. Gordeon, W J et al. The Relationship Between Limb Function and Radiographic Osteoarthrosis in Dogs with Stifle Osteoarthrosis. Veterinary Surgery 32:451-454 (2003)
12. Comblain F et al. Review of dietary supplements for the management of osteoarthritis in dogs in studies from 2004 to 2014. J. vet. Pharmacol. Therap. 39: 1-15 (2015)
13. Johnson M P et al. Comparative efficacy and safety of mavacoxib and carprofen in the treatment of canine osteoarthritis. Veterinary Record 176(11): 284 (2015)
14. Fleisch H. Introduction to bisphosphonates. History and functional mechanisms. Orthopade 36: 103-109 (2007)
15. Graham R et al. Bisphosphonates: The first 40 years. Bone 49: 2-19 (2011)
16 Rodan G A et al. Bisphosphonates: Mechanisms of Action. J. Clin. Invest. 97 (12): 2692-2696 (1996)
17. Beek v E R et al. Nitrogen-containing bisphosphonates inhibit isopentenyl pyrophosphate isomerase/Farnesyl pyrophosphate synthase activity with relative potencies corresponding to their antiresorptive potencies in vitro and in vivo. Biochemical and Biophysical Research Communications 255: 491-494 (1999)
18. Rodan G A. Alendronate: preclinical studies. Journal of Clinical Rheumatology 3 (2): S34-36 (1997)
19. Kanis J A et al. Rationale for the use of alendronate in osteoporosis. Osteoporosis Int. 5: 1-13 (1995)
20. G. Mohan et al. Pre-emptive, early, and delayed alendronate treatment in a rat model of knee osteoarthritis: effect on subchondral trabecular bone microarchitecture and cartilage degradation of the tibia, bone/cartilage turnover, and joint discomfort. Osteoarthritis and Cartilage 21: 1595-1604 (2013)
21. Little C B et al. Animal models of osteoarthritis. Current Rheumatology Reviews 4 (3): 1-8 (2008)
22. Teeple E et al. Animal models of osteoarthritis: Challenges of model selection and analysis. The AAPS Journal 15 (2): 438-446 (2013)
23. Meyer J M et al. Risedronate but not Alendronate slows disease progression in the guinea pig model of primary osteoarthritis. J Bone Miner Res 16: S305 (2001)
24. Davis A J et al. Are bisphosphonates effective in the treatment of osteoarthritis pain? A meta-analysis and systematic review. PLOS ONE 8 (9): e 72714 (2013)
25. Nishii t et al. Alendronate treatment for hip osteoarthritis: prospective randomized 2-year trial. Clin. Rheumatol. (2013) 32: 1759-1766 (2013)
26. Bingham C O et al. Risedronate decreases biochemical markers of cartilage degradation but does not decrease symptoms or slow radiographic progression in patients with medial compartment osteoarthritis of the knee. Arthritis & Rheumatism 54 (11): 3494-3507 (2006)
27. Oizumi T et al. Necrotic actions of nitrogen-containing bisphosphonates and their inhibition by clodronate, a non-nitrogen-containing bisphosphonate in mice: potential for utilization of clodronate as a combination drug with a nitrogen-containing bisphosphonate. Basic & Clinical Pharmacology & Toxicology 104: 384-392 (2009)
28. Price P A et al. Bisphosphonates alendronate and ibandronate inhibit artery calcification at doses comparable to those that inhibit bone resorption. Arterioscler Thromb Vasc Biol 21: 817-824 (2001)

1-9. (canceled)

10. A medicament compound comprising a salt of a nitrogen containing bisphosphonic acid (bisphosphonate) according to FIG. 1a 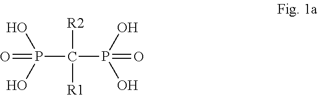
wherein R1 is independently selected from H and OH,
wherein R2 is independently selected from branched, unbranched, aromatic, non-aromatic cyclic, carbon comprising moieties having 1-12 carbon atoms and at least one of a primary amine, secondary amine, tertiary amine, or heterocycle comprising nitrogen, an analogue of said bisphosphonate, and combinations thereof,
for treating a naturally occurring disease/disorder in a mammal, comprising only the bisphosphonate or analogue thereof as active compound, wherein the disease/disorder is one or more of degenerative arthritis, degenerative joint disease, osteoarthrosis, arthritis, and Osteoarthritis, the mammal being selected from the orders of Perissodactyla, (Cet) Artiodactyla, and Carnivora,
wherein the medicament is a physiological liquid comprising a dosage of 0.01-1 mg/kg bisphosphonate,
wherein the medicament is for application once per 1-12 months, and
wherein the medicament is for sub-cutaneous or intravenous or intraperiotenal provision.

11. The medicament compound according to claim 10 for use in one of a horse, cattle, dog, or cat.

12. The medicament compound according to claim 10, wherein the carbon comprising moiety is selected from C₂-C₆ branched or un-branched alkyls, and 5- or 6-ring heterocyclic aromatic moieties comprising one or two nitrogens.

13. The medicament compound according to claim 10, wherein the bisphosphonate is selected from Pamidronate (FIG. 1c), Nerodronate (FIG. 1d), Olpadronate (FIG. 1e), amino-hydroxy-(n-hydroxy-oxido-phosphoryl)-alkyl-phosphonic acid according to FIG. 1b, Alendronate (FIG. 1i), Ibandronate (FIG. 1f), Risedronate (FIG. 1g), and Zoledronate (FIG. 1h).

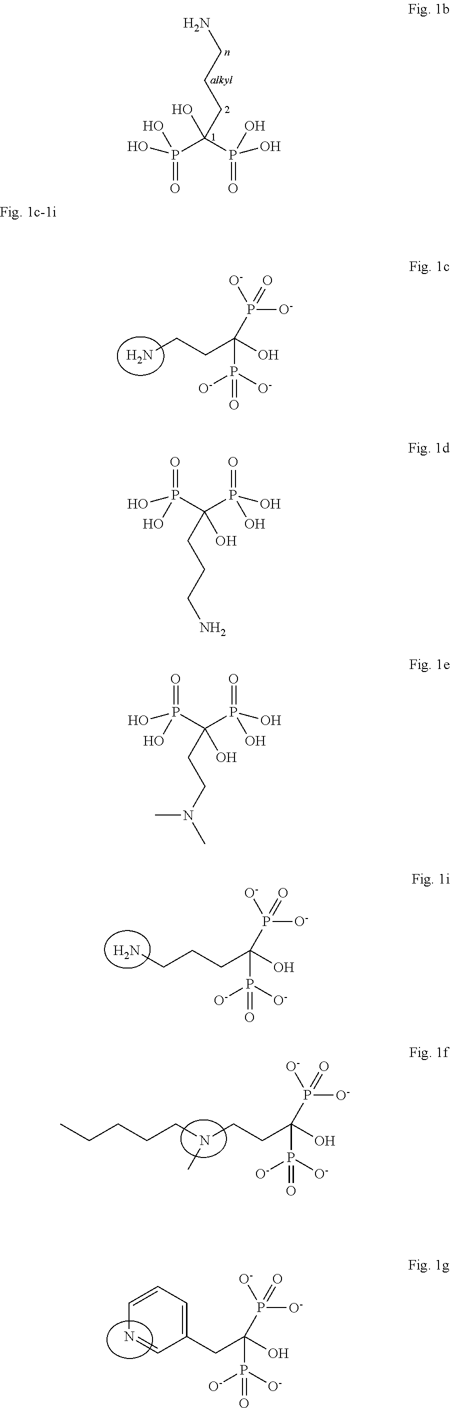

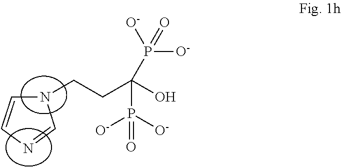 [SMILES: O=P([O-])([O-])C(O)(CCn1ccnc1)P(=O)([O-])[O-]]

14. The medicament compound according to claim 10, wherein the salt is an alendronate salt ([4-amino-1-hydroxy-1-(hydroxy-oxido-phosphoryl)-butyl]phosphonic acid trihydrate)[CAS number 121268-17-5].

15. The medicament compound according to claim 10, wherein the salt is a mono-valent salt, selected from Na—, K—, NH₄—, salts and combinations thereof.

16. The medicament compound according to claim 10, for the treatment of one or more of osteoarthritis in a joint, primary arthrosis, secondary arthrosis, development disorders, and disorders caused by hereditary and environmental influences, spondylosis deformans, deviating aggravation due to impairment, and inflammation.

17. The medicament compound according to claim 10, wherein the mammal is selected form Canidae, Felidae, Bovidae and Equidae.

18. The medicament compound according to claim 10, wherein the medicament is applied in a dosage of 0.1-0.3 mg per kg for Canidae and Felidae, and 0.01-0.25 mg, per kg for Bovidae and Equidae.

19. The medicament compound according to claim 10, wherein the medicament is applied as a single dose.
